(19) United States
(12) Patent Application Publication
(10) Pub. No.: US 2017/0014595 A1
(43) Pub. Date: Jan. 19, 2017

(54) CUSTOMIZED ANXIETY THERAPY DEVICE

(71) Applicant: Colleen Heath, Ortonville, MI (US)

(72) Inventor: Colleen Heath, Ortonville, MI (US)

(21) Appl. No.: 14/803,075

(22) Filed: Jul. 19, 2015

(51) Int. Cl. A61M 21/02

(52) U.S. Cl. CPC A61M 21/02; A61M 2021/0022

(57) ABSTRACT

A Customized Anxiety Therapy Device is designed to encourage tranquil, seated, repetitive behavior by Alzheimer's patients, dementia patients, and Autism Spectrum Disorder patients. The Customized Anxiety Therapy Device is constructed from a planar flexible material, upon which a plurality of differently colored and textured regions are durably fastened. A plurality of small tokens, which are easily handled and manipulated, are durably attached to the regions. A patient survey is included to identify tokens that are especially attractive or meaningful to the patient.

### FIELD OF INVENTION

This invention relates to the classes and sub-classes Education and Demonstration; and Exercise Devices. Specifically, this invention relates to the classes and sub-classes for developing or testing coordination and exercising the wrist and hand.

### BACKGROUND OF INVENTION

Alzheimer's disease is a progressive, degenerative brain disorder, in which amyloid plaques accumulate between the brain's neurons. Alzheimer's disease can lead to a loss of memory, changes in thinking, regression in language skill, and behavioral changes, often leading to dementia. The slide into dementia confuses and frightens those afflicted with Alzheimer's. Alzheimer's patients struggle to understand what is going on around them.

Repetitive activity is a hallmark of the dementia brought on by Alzheimer's disease. Often, a patient will make the same motion, repeat an activity, or get stuck asking the same line of questioning over long periods of time. Such behavior often takes a toll on the caregivers. Although the precise behavioral reasons for the repetitive behavior associated with Alzheimer's disease is unknown, many healthcare professionals conjecture that the patient is trying to self-sooth. That is, the patient is trying to relieve anxiety or fear by repeating an activity, motion, or line of question. The repetition is thought to provide comfort, security, and reassurance. An Alzheimer's patient's ability to interact with their environment is impaired by the memory problems associated with the disease, so the repetition may also be serving as a way for the patient to make sense of their situation and surrounding. Repetition may also be a result of memory loss and the person not being able to remember what they have done or said, or the answer they received to a question. Though it may confuse and concern the caregiver, the repetitive behavior seems to have meaning to the person with dementia. It is likely an attempt to enhance and maintain a sense of wellbeing and to ease distress.

A patient with Alzheimer's disease will often develop substantial stress and anxiety as a result of the progressive nature of the disease, and the deterioration in cognitive functions. The anxiety is often accompanied by sundowning or sunsetting. The term “sunsetting” or “sundowning” refers to a state of confusion at the end of the day and into the night. Sunsetting can cause a variety of behaviors, such as confusion, aggression or ignoring directions. Sunsetting can also lead to pacing or wandering. Clearly, regardless of the precise symptomology, sunsetting elevates the Alzheimer's patient already high level of anxiety. The exact cause of the sunsetting behavior is unknown. Often, families and medical professionals are at a loss for dealing with the anxiety caused by the disease, and by the anxiety associated with the sunsetting behavior.

Other afflictions share repetitive motion and sunsetting, such as autism spectrum disorder and non-Alzheimer's dementia. Caregivers of Alzheimer's patients, and patients with other forms of dementia or with autism spectrum disorder, are often emotionally exhausted by caring for their charges. Patients experiencing sunsetting, dementia, and autism spectrum disorder can be a hazard to themselves when they wander, and it can be difficult to get the patient to remain in one place. Many caregivers spend significant time corralling their charges. Additionally, a significant amount of time and effort is spent by caregivers in finding activities that will keep patients occupied and comforted.

One method of potentially soothing an Alzheimer's patient, or others, during an episode of sunsetting is to encourage the patient sooth themselves with a comforting repetitive behavior. Unfortunately, there are currently no devices on the market to serve this purpose.

What is really needed is a custom-made device, tailored to each specific patient that encourages a repetitive, soothing activity, to minimize the adverse behaviors associated with sunsetting, and to provide the patient with a distraction at other times. By using the patient's proclivity to perform repetitive activities, a suitable therapeutic device should be able to be fashioned with will encourage seated, tranquil, repetitive behavior. Such a custom-made device would present the patient with familiar and comforting repetitive activities. Additionally, such a custom-made device would be easily and comfortably held by the patient, remaining in the patient's lap while the patient is seated. Last, such a custom-made device would be constructed so that small pieces would not be lost or dropped. The market has not, yet, addressed this concern.

The current prior art has not established a suitable apparatus or device for encouraging comforting repetitive behavior in people afflicted with Alzheimer's disease, other forms of dementia, or on the autism spectrum. Additionally, there are no known therapies to adequately mitigate sunsetting behavior.

### SUMMARY OF THE INVENTION

The present invention fills a hole in the current prior art. The present invention is a therapeutic device, intended to distract and comfort Alzheimer's disease patients by encourage them to repeat a familiar motion or behavior. The present invention can be used with other patients, such as those with Autism Spectrum Disorder or dementia, who are prone to, and soothed by, repetitive motion. The therapeutic device is customized for each patient, using a patient survey, or another form of gathering individualized data about the patient. The information may be gathered from the patient, directly, if the patient has the mental faculties to do so. Otherwise the information can be gathered from the patient's family, loved ones or caregiver(s). Certain small items, or tokens, are known to pre-occupy Alzheimer's patients, and encourage repetitive, sedate, behavior. The survey is done to identify tokens that may hold meaning for the patient and that would engage the patient in repetitive activity.

The tokens may include, but is not limited to, pockets, keys, flaps, cords, straps, elastic loops, artificial hair, fabric flowers, plastic flowers, beaded strings, pendants, buttons, medals, buttons, zippers, small pad locks, wrist watches, pocket watches, rabbit feet, and squeezable balls. Although any small item holding meaning for the patient may be used as a token, most patients will be engaged by one or more of the foregoing list of tokens. Additionally, patients will often be encouraged to perform repetitive behavior based on texture or tactile stimulus.

The device is crafted specifically for an individual patient, and will include anywhere from two (2) to eight (8) tokens. The flexible planar material is overlaid, completely with materials of different textures and colors, called regions. The regions are durably fastened to the flexible planar material with stitching, gluing, or other suitable forms of fastening. The distinct regions are intended to focus the patient's attention on one token at a time, and encourage cyclical, repetitive behavior. The regions can be made from the same material as the flexible planar material, such as cotton (including denim), wool, ramie, silk, flax, leather, rayon, dacron (polyethylene terephthalate a.k.a. PET), polyester, polyethylene, polypropylene, nylon, or a blend or combination of one or more of the preceding. The regions can also be made from materials that encourage repetitive touching such as felt, denim, corduroy, suede, silk, satin, angora wool, camel hair, burlap, canvas, chintz, flannel, fleece, knit, leather, lycra, spandex, twill, tweed, ultrasuede, velvet, and velveteen.

The presentation of multiple tokens in multiple spatial regions also encourages patients to engage in a cycle of repetitive behavior, moving from one small token to the next. The tokens are durably fastened to the regions by stitching, gluing or other suitable forms of fastening. The flexible planar material provides a platform to present the tokens, in a comfortable way, to a patient in a seated position. The flexible planar material is sized so that it substantially covers the patient's lap.

### BRIEF DESCRIPTION OF THE DRAWINGS

The present invention, a Customized Anxiety Therapy Device, is illustrated with six (6) drawings.

FIG. 1 shows an isometric view of an embodiment of a Customized Anxiety Therapy Device.

FIG. 2 shows a reverse isometric view of an embodiment of a Customized Anxiety Therapy Device.

FIG. 3 shows an exploded isometric view of an embodiment of a Customized Anxiety Therapy Device.

FIG. 4 shows an isometric view of an alternative embodiment of a Customized Anxiety Therapy Device.

FIG. 5 shows an exploded isometric view of an alternative embodiment of a Customized Anxiety Therapy Device.

FIG. 6 shows an example embodiment of a survey used to customize the Customized Anxiety Therapy Device.

### DETAILED DESCRIPTION

The following descriptions are not meant to limit the invention, but rather to add to the summary of invention, and illustrate the Customized Anxiety Therapy Device.

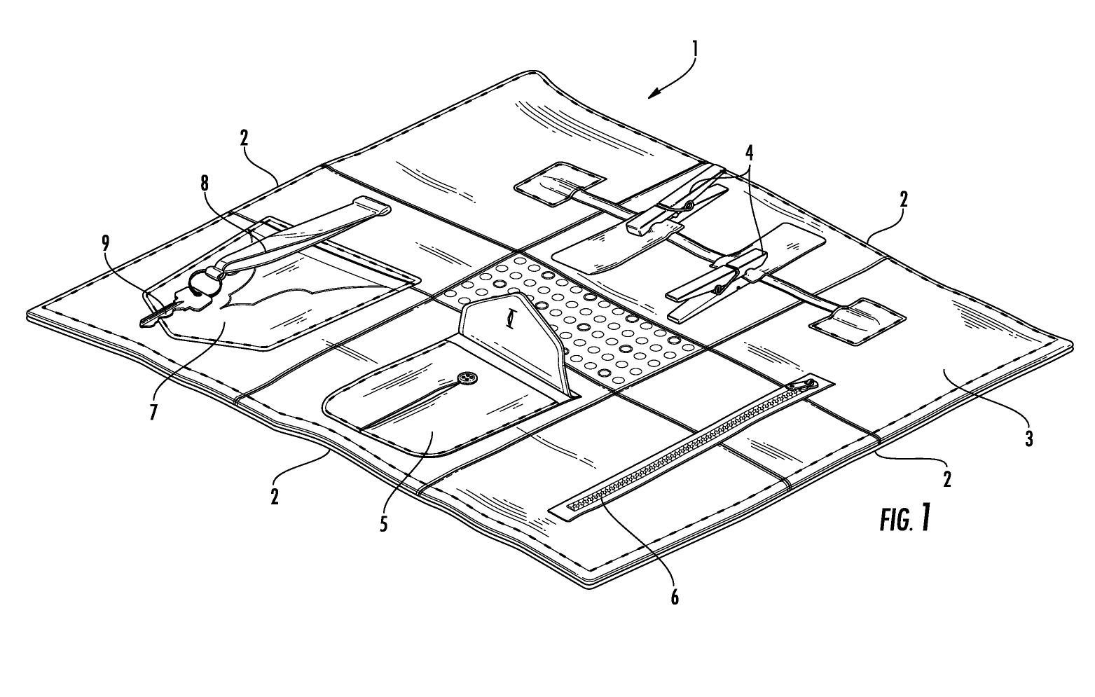

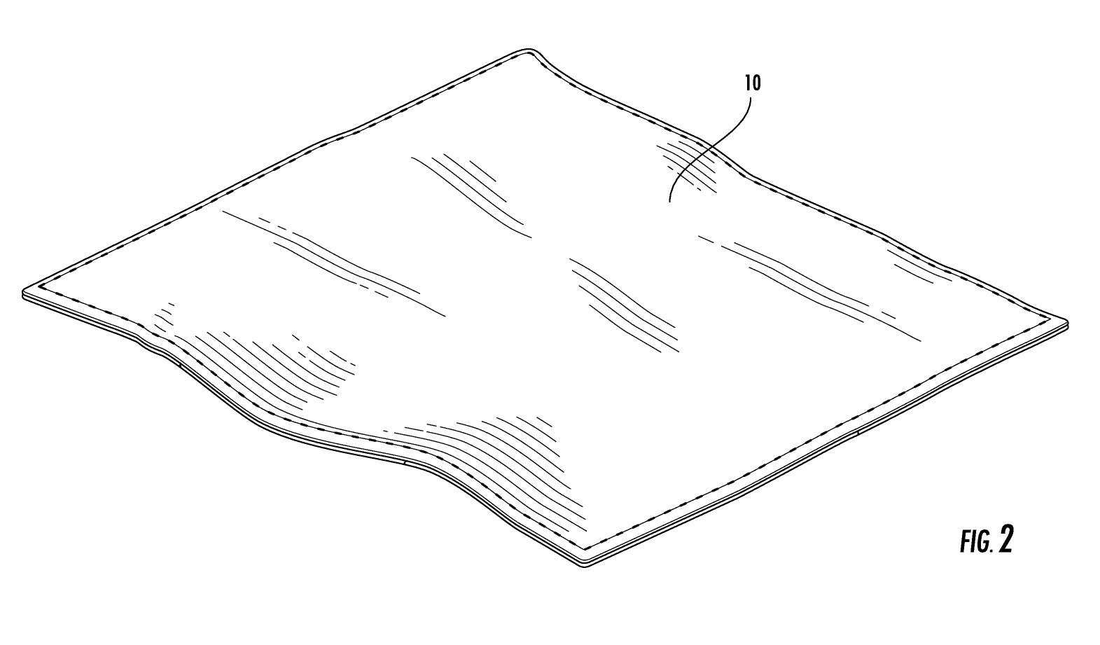

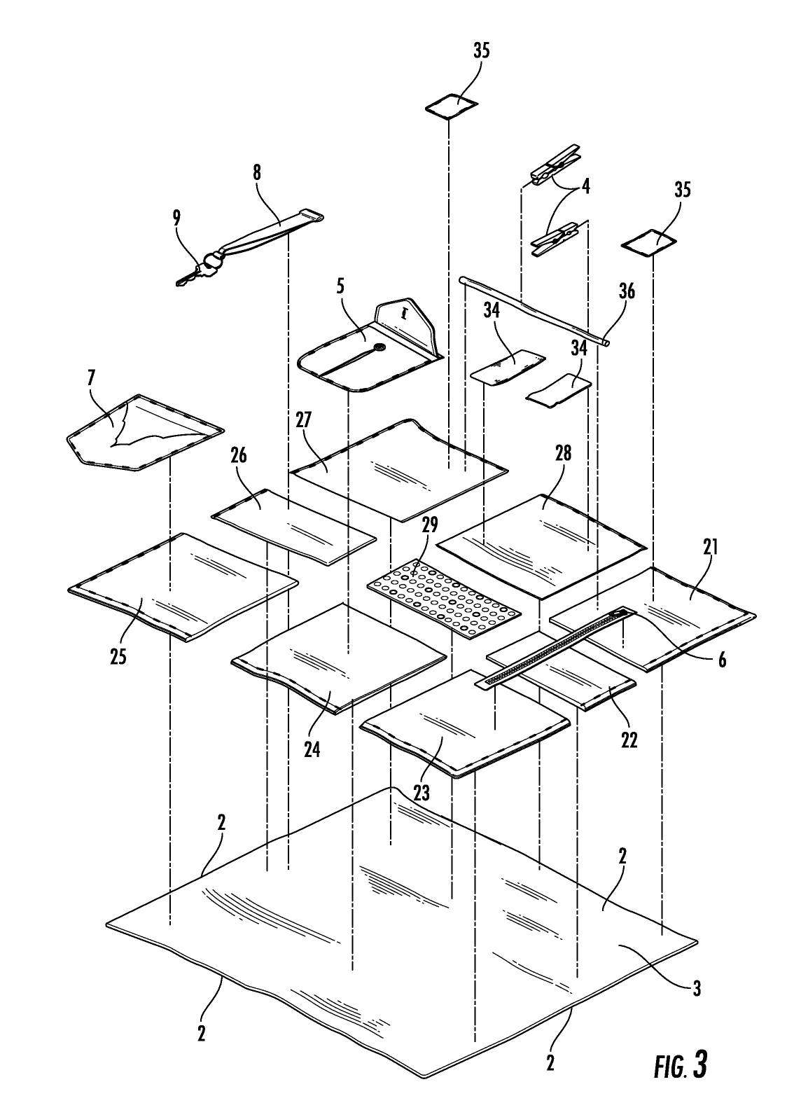

FIG. 2 is a reverse isometric of the Customized Anxiety Therapy Device, showing the back of the flexible planar material 10. FIG. 1 and FIG. 3 show an isometric view, and an isometric exploded view, respectively, of an embodiment of a Customized Anxiety Therapy Device 1. The present invention has four coplanar edges 2. Everything is mounted on top of the flexible planar material 3. The invention has a plurality of tokens 4, 5, 6, 7, 9. For this embodiment, the small tokens 4, 5, 6, 7, 9 are, specifically, clothes pins 4, pockets 5, 7, a zipper 6, and a key 9. The Customized Anxiety Therapy Device 1 is divided into regions 21, 22, 23, 24, 25, 26, 27, 28, 29. The regions 21, 22, 23, 24, 25, 26, 27, 28, 29 are flexible material with a variety of colors and textures, to provide contrast. The regions 21, 22, 23, 24, 25, 26, 27, 28, 29 completely overlay and cover the front of the flexible planar material 3. The regions 21, 22, 23, 24, 25, 26, 27, 28, 29 are durably secured to the front flexible planar material 3 using stitching, gluing, or another suitably durable fastener. The flexible planar material 3 is constructed from cotton (including denim), wool, ramie, silk, flax, leather, rayon, dacron (polyethylene terephthalate a.k.a. PET), polyester, polyethylene, polypropylene, nylon, or a blend or combination of one or more of the preceding. Likewise, the regions 21, 22, 23, 24, 25, 26, 27, 28, 29 can be constructed with cotton (including denim), wool, ramie, silk, flax, leather, rayon, dacron (polyethylene terephthalate a.k.a. PET), polyester, polyethylene, polypropylene, nylon, or a blend or combination of one or more of the preceding. Additionally, the regions 21, 22, 23, 24, 25, 26, 27, 28, 29 can be constructed from more tactile materials such as denim, corduroy, suede, silk, satin, angora wool, camel hair, burlap, canvas, chintz, flannel, fleece, knit, leather, lycra, spandex, twill, tweed, ultrasuede, velvet, and velveteen.

Some of the small tokens are secured directly to the regions (e.g., a pocket 5 is fastened directly to region 24; a zipper 6 is fastened directly across regions 23, 22, 21; a pocket 7 is fastened directly to region 25). Other tokens are durably fastened to the regions using a strap 8, a cord 36, or a tab 34, or other similar fastening method. For example, the clothes pins 4 are designed to fasten to the cord 36 and tabs 34. The cord 36 is fastened to the regions 21, 27 with patches 35. The tabs 34 are fastened to the region 28 at one end and are free at the other.

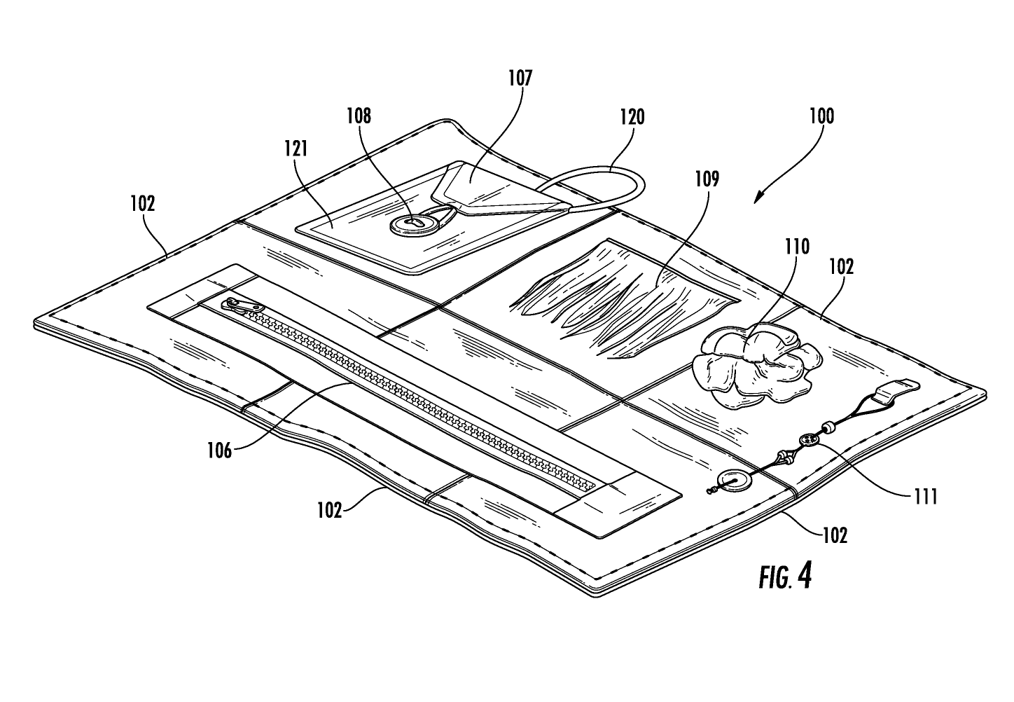

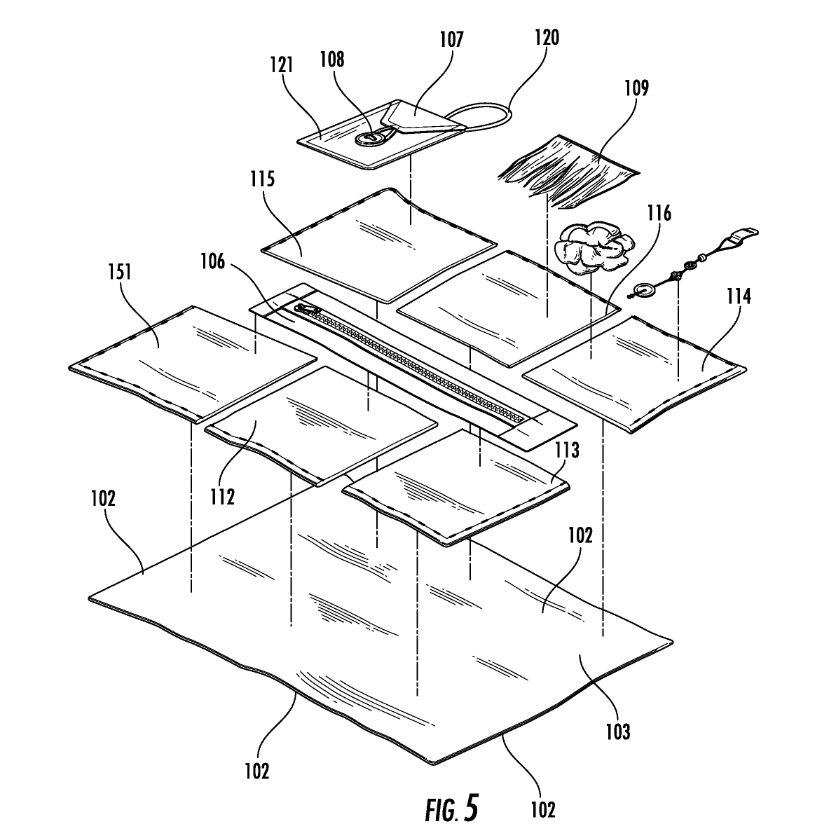

FIG. 4 and FIG. 5 show an isometric view, and an isometric exploded view, respectively, of an alternative embodiment of a Customized Anxiety Therapy Device 100. The present invention has four coplanar edges 102. Everything is mounted on top of the flexible planar material 103. The invention has a plurality of tokens 106, 107, 108, 109, 110, 111, 120, 121. For this embodiment, the small tokens 106, 107, 108, 109, 110, 111, 120, 121 are, specifically, a zipper 106; a pocket 121 with a flap 107, a pendant 108, and a loop of elastic 120; a swatch of artificial hair 109; a fabric flower 110; and a beaded string 111. The Customized Anxiety Therapy Device 100 is divided into regions 112, 113, 114, 115, 116, 151. The regions 112, 113, 114, 115, 116, 151 are flexible material with a variety of colors and textures, to provide contrast. The regions 112, 113, 114, 115, 116, 151 completely overlay and cover the front flexible planar material 103. The regions 112, 113, 114, 115, 116, 151 are durably secured to the front flexible planar material 103 using stitching, gluing, or another suitably durable fastener. The flexible planar material 103 is constructed from cotton (including denim), wool, ramie, silk, flax, leather, rayon, dacron (polyethylene terephthalate a.k.a. PET), polyester, polyethylene, polypropylene, nylon, or a blend or combination of one or more of the preceding. Likewise, the regions 112, 113, 114, 115, 116, 151 can be constructed from cotton (including denim), wool, ramie, silk, flax, leather, rayon, dacron (polyethylene terephthalate a.k.a. PET), polyester, polyethylene, polypropylene, nylon, or a blend or combination of one or more of the preceding. Additionally, the regions 112, 113, 114, 115, 116, 151 can be constructed from more tactile material such as denim, corduroy, suede, silk, satin, angora wool, camel hair, burlap, canvas, chintz, flannel, fleece, knit, leather, lycra, spandex, twill, tweed, ultrasuede, velvet, and velveteen.

Some of the small tokens are secured directly to the regions (e.g., the zipper 106 is fastened directly across regions 112, 113, 151; artificial hair 109 is fastened directly to a region 116; a fabric flower 110 is fastened directly to a region 114; and a beaded string 111 is fastened directly to a region 114). A flap 107 is attached to a pocket 121, with a pendant 108 attached to the flap. A loop of elastic 120 is attached to the pocket 121 and flap 107.

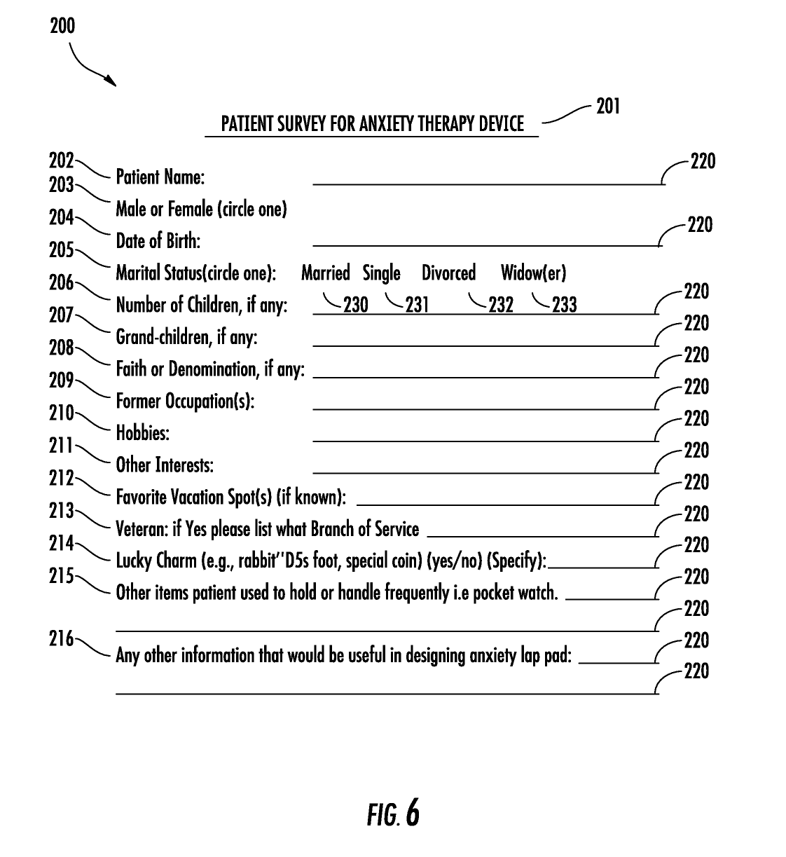

FIG. 6 shows a representative sample of a patient survey 200 to develop customization ideas for the Customized Anxiety Therapy Device 1, 100. The survey title 201 is at the top of the paper. Identifying information, such as patient name 202, sex (male or female) 203, and date of birth 204 begin the survey. Information about the patient's family is next: marital status 205, number of children 206, number of grand-children 207. Questions about the patient's faith 208, vocation(s) 209, avocation(s) 210, 211, favorite vacation spot(s) 212, veteran status 213, and lucky charms 214 are asked. Last, questions about potential tokens and other useful information for designing anxiety device are asked 215, 216. Across from most questions 202, 204, 206, 207, 208, 209, 210, 211, 212, 213, 214, 215, 216 is a space to record information 220. In the case of the question about sex 203, the appropriate selections to circle are given 203. In the case of the question about marital status 205, the appropriate marital status 230, 231, 232, 233 are presented as choices to circle.

The information from the patient survey 200 is used to inform the creation of a Customized Anxiety Therapy Device 1, 100. Vocations 209 such as florist, tailor, beautician would be associated with tokens such as artificial flowers 110; pockets 121, 5, 7; and artificial hair 109. A parent or grandparent would be associated with buttoning pockets 105 or zippers 106, 6. A businessman may also associate with a variety of pockets 121, 5, 7. A favorite pendant 108 may be used. A person who carried keys for work or to drive may be given a key on a strap 8, 9. The patient can also be presented with a variety of tokens 4, 5, 6, 7, 9, 106, 107, 108, 109, 110, 111, 120, 121, to assess their interest in each. Likewise, the patient can be presented with material of different colors and textures to determine their preferences for regions 21, 22, 23, 24, 25, 26, 27, 28, 29, 112, 113, 114, 115, 116, 151. After presenting a patient with tokens 4, 5, 6, 7, 9, 106, 107, 108, 109, 110, 111, 120, 121 and material of different colors and textures for regions 21, 22, 23, 24, 25, 26, 27, 28, 29, 112, 113, 114, 115, 116, 151, a Customized Anxiety Therapy Device 1, 100 is created. If the patient engages a Customized Anxiety Therapy Device 1, 100, and exhibits the desired tranquil and content repetitive behavior, the process is over. If the patient is not suitably engaged with a Customized Anxiety Therapy Device 1, 100, different tokens 4, 5, 6, 7, 9, 106, 107, 108, 109, 110, 111, 120, 121 and materials of different colors and textures for regions 21, 22, 23, 24, 25, 26, 27, 28, 29, 112, 113, 114, 115, 116, 151 can be substituted.

We claim:

1. A Customized Anxiety Therapy Device comprising a planar flexible material base; a plurality of colored and textured regions, constructed from flexible material and durably attached to said planar flexible material base; a plurality of tokens durably attached to said plurality of colored and textured regions, wherein the tokens, though durably attached, are free to be used and moved about said Custom Anxiety Therapy Device; said Customized Anxiety Therapy Device being designed and constructed using a patient survey; and said Customized Anxiety Therapy Device being of a size and shape to cover most an adult's seated lap.

2. The invention in claim 1, wherein said planar flexible material base has at least four (4) coplanar edges.

3. The invention in claim 2, wherein the planar flexible material base is constructed from material comprising cotton (including denim), wool, ramie, silk, flax, leather, rayon, dacron (polyethylene terephthalate a.k.a. PET), polyester, polyethylene, polypropylene, nylon, or a blend or combination of one or more of the preceding.

4. The invention in claim 1, wherein said plurality of colored and textured regions completely overlays and covers said planar flexible material base.

5. The invention in claim 4, wherein said plurality of colored and textured regions are constructed from material comprising cotton (including denim and corduroy), wool, ramie, silk, flax, leather, rayon, dacron (polyethylene terephthalate a.k.a. PET), polyester, polyethylene, polypropylene, nylon, suede, silk, satin, angora wool, camel hair, burlap, canvas, chintz, flannel, fleece, knit, leather, lycra, spandex, twill, tweed, ultrasuede, velvet, velveteen or a blend or combination of one or more of the preceding.

6. The invention in claim 1, wherein the plurality of tokens are selected from a set consisting essentially of pockets, keys, flaps, cords, straps, elastic loops, artificial hair, fabric flowers, plastic flowers, beaded strings, pendants, buttons, medals, buttons, zippers, small pad locks, wrist watches, pocket watches, rabbit feet, and squeezable balls.

7. The invention in claim 1, wherein said patient survey comprises questions about name, gender, date of birth, marital status, children, grand-children, avocations, vocations, veteran status, lucky charms, and religious affiliation.

8. The invention in claim 1, wherein said planar flexible material base has at least four (4) coplanar edges and wherein said planar flexible material base is constructed from material comprising cotton (including denim), wool, ramie, silk, flax, leather, rayon, dacron (polyethylene terephthalate a.k.a. PET), polyester, polyethylene, polypropylene, nylon, or a blend or combination of one or more of the preceding; wherein said plurality of colored and textured regions completely overlays and covers said planar flexible material base and wherein said plurality of colored and textured regions are constructed from material comprising cotton (including denim and corduroy), wool, ramie, silk, flax, leather, rayon, dacron (polyethylene terephthalate a.k.a. PET), polyester, polyethylene, polypropylene, nylon, suede, silk, satin, angora wool, camel hair, burlap, canvas, chintz, flannel, fleece, knit, leather, lycra, spandex, twill, tweed, ultrasuede, velvet, velveteen or a blend or combination of one or more of the preceding; wherein the plurality of tokens are selected from a set consisting essentially of pockets, keys, flaps, cords, straps, elastic loops, artificial hair, fabric flowers, plastic flowers, beaded strings, pendants, buttons, medals, buttons, zippers, small pad locks, wrist watches, pocket watches, rabbit feet, and squeezable balls; and wherein said patient survey comprises questions about name, gender, date of birth, marital status, children, grand-children, avocations, vocations, veteran status, lucky charms, and religious affiliation.
